(19) United States
(12) Patent Application Publication
(10) Pub. No.: US 2026/0337678 A1
(43) Pub. Date: Oct. 29, 2026

(54) Method for confirming the sequence of a nucleic acid

(71) Applicant: Thermo Fisher Scientific (Bremen) GmbH, Bremen (DE)

(72) Inventors: Ralf Günter Hartmer, Hamburg (DE); Kerstin Strupat, Harpstedt (DE)

(73) Assignee: Thermo Fisher Scientific (Bremen) GmbH, Bremen (DE)

(21) Appl. No.: 19/548,940

(22) Filed: Feb. 24, 2026

(30) Foreign Application Priority Data
Feb. 25, 2025 (GB) GB2502730.1

(51) Int. Cl. C12Q 1/6872; H01J 49/00; H01J 49/16; H01J 49/42

(52) U.S. Cl. CPC C12Q 1/6872; H01J 49/0036; H01J 49/164; H01J 49/4215

(57) ABSTRACT

A method for confirming the sequence of a nucleic acid There is provided a method for confirming the sequence of a nucleic acid, the method comprising: (i) hydrolytic cleavage of predetermined phosphodiester bonds in the nucleic acid to produce a plurality of oligonucleotides; (ii) ionising the plurality of oligonucleotides to provide a gaseous phase of ionised oligonucleotides, wherein a majority of the ionised oligonucleotides are singly charged; (iii) isolating the ionised oligonucleotides using a plurality of mass to charge ratio (m/z) windows; (iv) detecting the isolated ionised oligonucleotides of each m/z window using a high-resolution mass spectrometer (HRMS) having a resolving power of greater than 30,000 at m/z 200, thereby obtaining a mass spectrum; (v) confirming the sequence of the nucleic acid by comparison of the mass spectrum obtained in step (iv) against a mass spectrum determined in silico following the same hydrolytic cleavage of the nucleic acid as in step (i), wherein the confirmation comprises comparison of the signal intensities of one or more mass envelopes resulting from the ionised oligonucleotides.

### TECHNICAL FIELD

The present invention relates to a method for confirming the sequence of a nucleic acid by mass spectrometry. More particularly, a method in which the sequence of the nucleic acid is confirmed by comparison of a mass spectrum of a plurality of oligonucleotides obtained therefrom by hydrolytic cleavage against a mass spectrum determined in silico following the same hydrolytic cleavage of the nucleic acid.

### BACKGROUND

The endonucleic digestion of nucleic acids such as RNA has been well-known and there have been reports which describe the MALDI MS analysis of RNA after the G-specific digestion with RNase T1. For example, Nucleic Acids Research 2003, 31(9), e47 “RNase T1 mediated base-specific cleavage and MALDI-TOF MS for high-throughput comparative sequence analysis” provides a method for comparative sequence analysis which comprises a homogeneous in vitro transcription/RNase cleavage system with the accuracy and data acquisition speed of matrix-assisted laser desorption/ionisation coupled with linear axial time-of-flight mass spectrometry (MALDI-TOF MS). The feasibility study shows capability for “rapid automated comparative sequence analysis for PCR products up to 1 kb in length”.

More recently, the analysis of mRNA has become extremely important for vaccine development. For example, clinical and commercial manufacturing process changes are common during mRNA vaccine production, and analytical techniques are required to demonstrate product comparability for pre- and post-change batches. Scientific Reports 2023, 13, 9038 “Oligonucleotide mapping via mass spectrometry to enable comprehensive primary structure characterisation of an mRNA vaccine against SARS-CoV-2” describes oligonucleotide mapping via liquid chromatography with UV detection coupled to tandem mass spectrometry (LC-UV-MS/MS). It is said that in a single method “oligonucleotide mapping readouts include a highly reproducible and completely annotated UV chromatogram with 100% maximum sequence coverage, and a microheterogeneity assessment of 5′ terminus capping and 3′ terminus poly(A)-tail length”.

In Int. J. Mol. Sci. 2023, 24, 14691 “Quantification of Oligonucleotides Using Tandem Mass Spectrometry with Isobaric Internal Standards”, there is disclosed a quantification method using liquid chromatography coupled with tandem mass spectrometry (LC-MS/MS) by applying an isobaric oligonucleotide standard to quantify oligonucleotides. Specifically, it is said that the method utilises “multiple reaction monitoring (MRM) of a single precursor ion and two distinct daughter ions for quantification”.

WO2018/081462A1 discloses a method for determining the presence of an RNA in a mRNA sample, by determining a signature profile of the mRNA sample, comparing the signature profile to a known signature profile for a test mRNA, identifying the presence of an RNA in the mRNA sample based on a comparison with the known signature profile for the test mRNA which may be determined by LC-MS/MS mRNA sequence mapping.

Despite these developments in the prior art, there remains a need for a method which provides for a fast and accurate confirmation of a nucleic acid sequence. The present invention was developed with the aim of addressing such problems in the prior art, or to at least provide a commercially useful alternative thereto.

### SUMMARY

A first aspect of the present invention provides a method for confirming the sequence of a nucleic acid, the method comprising:
(i) hydrolytic cleavage of predetermined phosphodiester bonds in the nucleic acid to produce a plurality of oligonucleotides;
(ii) ionising the plurality of oligonucleotides to provide a gaseous phase of ionised oligonucleotides, wherein a majority of the ionised oligonucleotides are singly charged;
(iii) isolating the ionised oligonucleotides using a plurality of mass to charge ratio (m/z) windows;
(iv) detecting the isolated ionised oligonucleotides of each m/z window using a high-resolution mass spectrometer (HRMS) having a resolving power of greater than 30,000 at m/z 200, thereby obtaining a mass spectrum;
(v) confirming the sequence of the nucleic acid by comparison of the mass spectrum obtained in step (iv) against a mass spectrum determined in silico following the same hydrolytic cleavage of the nucleic acid as in step (i), wherein the confirmation comprises comparison of the signal intensities of one or more mass envelopes resulting from the ionised oligonucleotides.

In another aspect, the present invention allows for the comparison of two experimentally acquired mass spectra, such a method can be particularly useful for identifying functionalisation of the nucleic acid against the parent (i.e. a reference) nucleic acid, or any other modifications (e.g. changes in sequence) against the parent nucleic acid. Thus, the further aspect provides a method for confirming the sequence of a nucleic acid, the method comprising:
(i) hydrolytic cleavage of predetermined phosphodiester bonds in the nucleic acid to produce a plurality of oligonucleotides;
(ii) ionising the plurality of oligonucleotides to provide a gaseous phase of ionised oligonucleotides, wherein a majority of the ionised oligonucleotides are singly charged;
(iii) isolating the ionised oligonucleotides using a plurality of mass to charge ratio (m/z) windows;
(iv) detecting the isolated ionised oligonucleotides of each m/z window using a high-resolution mass spectrometer (HRMS) having a resolving power of greater than 30,000 at m/z 200, thereby obtaining a mass spectrum;
(v) confirming the sequence of the nucleic acid by comparison of the mass spectrum obtained in step (iv) against a mass spectrum obtained by duplicating steps (i) to (iv) with a reference nucleic acid, wherein the confirmation comprises comparison of the signal intensities of one or more mass envelopes resulting from the ionised oligonucleotides.

### DESCRIPTION OF THE DRAWINGS

The present invention will now be described further with reference to the following exemplary non-limiting Figures, in which:

FIG. 1 illustrates the in silico calculation of the signal intensities of the plurality of oligonucleotides obtained from digestion of both the Comirnaty BNT162b2 Original DS vaccine and BNT162b2s05 Omicron variant.

FIG. 2 illustrates the in silico calculation of the signal intensities of the plurality of oligonucleotides obtained from digestion of both the Comirnaty BNT162b2 Original DS vaccine and BNT162b2s04 Delta variant.

FIG. 3 provides a close-up view of the data of FIG. 1 within the m/z range of 900 to 1,400.

FIG. 4 provides a close-up view of the data of FIG. 1 within the m/z range of 1,500 to 2,000.

FIG. 5 provides a close-up view of the data of FIG. 1 (and FIG. 4) within the m/z range of 1,880 to 1,950.

FIG. 6 provides a close-up view of the data of FIG. 1 within the m/z range of 2,000 to 5,000.

FIG. 7 provides a close-up view of the data of FIG. 1 within the m/z range of 4,600 to 5,400.

FIG. 8 provides a close-up view of the data of FIG. 2 within the m/z range of 4,600 to 5,400.

FIG. 9 provides a close view of FIG. 1 (and FIG. 6) within the m/z range of 2,190 to 2,290.

### DETAILED DESCRIPTION

The present disclosure will now be described further. In the following passages, different aspects/embodiments of the disclosure are defined in more detail. Each aspect/embodiment so defined may be combined with any other aspect/embodiment or aspects/embodiments unless clearly indicated to the contrary. In particular, any feature indicated as being preferred or advantageous may be combined with any other feature or features indicated as being preferred or advantageous.

The method allows for the confirmation of the sequence of a nucleic acid. As will be appreciated, such a method therefore allows for a positive confirmation (i.e. a positive result) as to the identity and therefore the sequence of the nucleic acid being tested, but also a negative confirmation (i.e. a negative result) in which the identity and sequence of the nucleic acid tested is not that which it was compared to.

The method comprises a first step (i) in which predetermined phosphodiester bonds in the nucleic acid are hydrolytically cleaved to produce a plurality of oligonucleotides. As will be appreciated, nucleic acids are large biomolecules comprised of nucleotides in which the sugars and the phosphates are alternatively connected by phosphodiester bonds providing the nucleic acid with a characteristic sequence. The hydrolytic cleavage may generally be performed by enzymatic digestion with a nuclease, specifically an endonuclease. The essential aspect of the first step is that the cleavage of the phosphodiester bonds is predetermined so as to allow for an appropriate in silico determination of the expected mass spectrum. Endonucleases, such as restriction enzymes, cleave phosphodiester bonds within the polynucleotide chain of nucleic acids (e.g. DNA or RNA) at specific sites (i.e. cutting sites). That is, a given endonuclease will cleave phosphodiester bonds at known predetermined positions within a sequence following binding at a particular recognition sequence or recognition site, or simply, the step comprises sequence specific cleavage. Equally, by comparison with the computed spectrum associated with a given nucleic acid, differences in the experimental spectrum may be used to determine sequence variations between nucleic acids.

As described above, there is a particular drive for the analysis of mRNA for vaccine development, and the present invention is ideal for the confirmation of an mRNA sequence. mRNA refers to messenger RNA which is a single-stranded molecule of RNA created by transcription. An RNA polymerase enzyme first creates a so-called pre-mRNA by transcription of DNA that may still contain introns (i.e. intragenic regions, which are nucleotide sequences that do not directly code for proteins). Post-transcription processes, such as RNA splicing, leaves only the exons (i.e. exogenic regions, which are nucleotide sequences that do code for proteins), this constituting the mRNA. mRNA is generally composed of a 5′ cap, a 5′ untranslated region, a coding sequence, a 3′ untranslated region and a polyadenylate tail (poly-A tail).

As large biomolecules, the nucleic acid with generally have at least 100 nucleotides, preferably at least 1,000 nucleotides. There is no specific upper limit, though RNAs tend to have no more than a few thousand nucleotides (e.g. up to 4,000 nucleotides), whereas DNA may have hundreds of millions or billions (e.g. up to 10 billion). The use of a high resolution mass spectrometer as described herein permits accurate determination when compared with techniques used in the prior art (e.g. axial ToF).

Examples of suitable sequence specific endonucleases include EcoRI, HindIII, HaeIII BamHI, MazF, and RNase enzymes such as RNase A, RNase V, RNase T1, RNase U2. By way of example, RNase A cleaves specifically at Y\hat{ }N (where Y=C or T/U), RNase V cleaves specifically at C\hat{ }N, RNase T1 cleaves specifically at G\hat{ }N, and RNase U2 cleaves specifically at R\hat{ }N (where R=G or A), and where N=any base. Many of such suitable endonucleases are commercially available. In particularly preferred embodiments of the present invention, step (i) comprises digestion of the nucleic acid with the G-specific RNase T1 to produce a plurality of oligonucleotides. As such, the digestion of a given nucleic acid is predictable, and as described herein, the method of the first aspect involves a comparison with computed data following an equivalent process (i.e. in silico). As will be appreciated, the present method does not explicitly require the in silico calculation be performed by the same user as that performing the experimental analysis of the nucleic acid (i.e. the sample preparation and mass spectrometry), but may simply comprise comparison of the mass spectrum obtained in step (iv) against a database of mass spectra, wherein each mass spectrum has been determined in silico following the same endonucleolytic digestion in step (i) of a further nucleic acid (and a positive confirmation may therefore be ascertained where the nucleic acids are the same). Such a process may be used to determine the identity and sequence of an otherwise unknown nucleic acid sample. However, the method may be used for quality control in, for example, nucleic acid synthesis, whereby the mass spectrum experimentally obtained is compared with that determined in silico for the expected, or targeted, synthetic product.

The hydrolytic cleavage produces a plurality of oligonucleotides. Oligonucleotides are oligomers of nucleotides, and are generally short chain sequences of nucleotides. Following hydrolytic cleavage, these oligomers which make up the entire nucleic acid have different lengths based on the number of nucleotides, with a majority comprising from 2 up to about 25 nucleotides, particularly for RNA. The length of the oligonucleotide is often suffixed by the term “mer”. A plurality of oligonucleotides refers to at least two fragments of the nucleic acid, though generally there will be significantly more (e.g. more than 100, more than 200, more than 300, or more), with an array of unique oligonucleotides across many lengths (often at least 2 to 14 nucleotides), as well as unique oligonucleotides having the same length. As described further herein, shorter chain oligonucleotides are statistically more likely to be repeated due to their natural presence in the nucleic acid and thus have a larger repeat abundance, whereas longer chains, particularly 8mers and above, or even 6mers and above, are typically all unique fragments. Many fragments may still have the same exact mass (i.e. isobaric oligonucleotides which are structural isomers).

When compared to sequencing a polypeptide, where more than 22 amino acids exist in nature and can be combined to contribute to different molecular masses of peptides, the variety in the individual building blocks of the oligonucleotides is significantly smaller. In general, the combination of DNA or RNA only needs four nucleotides resulting in a less complex mixture of oligonucleotide, particularly if a hydrolytic cleavage of the phosphodiester bond of the DNA or RNA is applied. The four nucleobases in DNA are guanine (G), adenine (A), cytosine (C), and thymine (T); in RNA, uracil (U) is used in place of thymine. Further nucleobases are known. The N-methylpseudouridine (m1ψ) plays nowadays an important role in the synthesis of mRNA vaccines as a replacement for natural uracil, and the present invention is particularly beneficial in the analysis of such mRNA.

Suitable conditions for performing step (i) will be readily apparent to those skilled in the art so as to provide a substantially complete digestion of the nucleic acid, with various protocols well-known in the art. A reaction digest may involve the addition of 1 unit of restriction enzyme to 1 μg of nucleic acid in a 50 μL reaction volume for about 1 hour at 37 °C. Suitable digestion protocols are described in, for example, Anal. Chem. 2022, 94, 20, 7339-7349. More typically, 5 to 50 units of the restriction enzyme are used per μg of nucleic acid. The digestion reaction is typically performed with a buffer in aqueous solution, and may include a chelating agent. The digestion reaction is typically performed at a temperature of about 30 °C to 50 °C (i.e. incubated) and may be incubated for from 30 minutes to 90 minutes. The restriction enzyme may be immobilised on magnetic beads which can be collected and removed from the reaction mixture following digestion.

The method further comprises a second step (ii) of ionising the plurality of oligonucleotides to provide a gaseous phase of ionised oligonucleotides, wherein a majority of the ionised oligonucleotides are singly charged. Preferably at least 90% by weight, and more preferably substantially all, of the ionised oligonucleotides are singly charged. This avoids interference within the spectrum from multiply charged ions and facilitates the accurate determination and assignment of unit signal intensity for the comparison of quantised signal intensities.

Suitable soft ionisation techniques include MALDI (matrix-assisted laser desorption ionisation), photoionisation and DART (direct analysis in real time). In preferred embodiments, the plurality of oligonucleotides is ionised in step (ii) by atmospheric pressure MALDI (AP-MALDI), more preferably liquid atmospheric pressure MALDI (LAP-MALDI). MALDI (in particular (L)AP-MALDI as opposed to the more conventional vacuum MALDI) is a high-throughput mass spectrometry technique which produces singly charged ions, and thus ion peaks which are well separated in the mass space, which, when coupled with a high resolution mass spectrometer as described herein, allows for rapid sequence confirmation of many samples. Each sample may be analysed in less than one minute, allowing for hundreds of analyses per day. For example, the method may be performed conveniently directly from a multiwell (microtiter) plate, such as a 96 or 384 well plate. Moreover, the inventors believe that atmospheric pressure MALDI is better suited for oligonucleotide analysis because the thermal cooling effect of the MALDI plume is less prone to being dominated by in source decomposition which is known to be an issue for MALDI ionisation in high vacuum.

The method further comprises a third step (iii) of isolating the ionised oligonucleotides using a plurality of mass to charge ratio (m/z) windows. The ionisation of the plurality of oligonucleotides provides a map of ions which are well separated in the mass space by the individual length (and therefore weight) of the oligonucleotide. Only the area of calculated masses of the oligonucleotides are targeted which allows for a “binning” of the mass spectrum into windows of the nucleotide length (i.e. isolation), and subsequent detection of one window at a time.

That is, mass isolation in mass spectrometry is the process of separating ions by their m/z ratio, and this may be done using electric and/or magnetic fields. A mass filter is arranged downstream of the ion source (e.g. the AP-MALDI) and is configured to receive ions from the ion source. The mass filter is configured to filter the received ions according to their m/z ratio, and the mass filter may be configured such that received ions having m/z within an m/z transmission window (or “isolation window”) of the mass filter are onwardly transmitted by the mass filter, while received ions having m/z outside the m/z transmission window are attenuated by the mass filter (i.e. are not onwardly transmitted by the mass filter). The width and/or the centre m/z of the transmission window may be controllable (variable), such as by suitable control of RF and/or DC voltage(s) applied to electrodes of the mass filter. Thus, for example, the mass filter may be operable in a transmission mode of operation, whereby most or all ions within a relatively wide m/z window are onwardly transmitted by the mass filter, and a filtering mode of operation, whereby only ions within a relatively narrow m/z window (centred at a desired m/z) are onwardly transmitted by the mass filter. The mass filter can be any suitable type of mass filter, such as a quadrupole mass filter.

The mass filter can be used to select only ions of interest (based on the predicted m/z binning) and to filter out other ions such as background ions. As such, it is preferred that each of the plurality of m/z windows spans a range of m/z ratios which encompasses oligonucleotides of a single specific length. As such, it is generally the case, and therefore preferred, that each window does not overlap. The focussing of detection within pre-determined m/z windows reducing the detection and analysis time.

Preferably, between the isolation and detection steps (iii) and (iv), the method comprises accumulating the isolated ionised oligonucleotides within an ion trap arranged upstream of the mass analyser (and may be referred to as a secondary ion trap where combined with a primary ion trap mass analyser), and further comprises periodically passing the accumulated ionised oligonucleotides from the ion trap to the mass analyser for detection of the ionised oligonucleotides. GB2618673A discloses methods of controlling the number of ions accumulated in an ion trap, and describes methods therein which are suitable for the accumulation of isolated ionised oligonucleotides, in particular using ion traps such as a linear ion trap or a curved linear ion trap (C-trap), as further described in, for example, WO2008/081334A2. The ion trap may be used to cool the accumulated ions prior to injecting them into the mass analyser. The ion trap may be configured such that ions can be ejected in a pulsed manner. By accumulating the isolated ions within an ion trap, the trap can be filled up with mostly/only ions of interest and the quantification of these ions by mass analysis can be improved.

The ion trap may include an entrance lens or gate and an exit lens or gate. The entrance lens can be operated in an open mode, in which ions (from the ion source) can pass the entrance lens and enter the ion trap, or a closed mode in which ions (from the ion source) cannot pass the entrance lens and do not enter the ion trap. When the entrance lens is operated in its closed mode, ions already within the ion trap will not be able to leave the ion trap via the entrance lens. Similarly, the exit lens can be operated in an open mode, in which ions can pass the exit lens and leave the ion trap, or a closed mode in which ions cannot pass the entrance lens and do not leave the ion trap. The entrance lens (the exit lens) can be closed or opened by applying a suitable voltage to the entrance lens (to the exit lens).

Ions from the ion source can be accumulated in the ion trap by operating the exit lens in its closed mode, while operating the entrance lens in its open mode. After a desired ion fill time of ions into the ion trap, the entrance lens can be closed (by altering the voltage applied to the entrance lens) such that ions cannot pass out of the trap and such that ions from the ion source can no longer enter the ion trap. In some embodiments, more accurate gating of the incoming ion beam into the trap can be provided by a lens or gate electrode within one or more ion transfer stage(s) upstream of the secondary ion trap. In these embodiments, the lens or gate electrodes may be configured such that the transmission of ions into the ion trap can be switched on or off.

Thus, the mass spectrometer may be configured such that ions can be accumulated in the secondary ion trap with an adjustable accumulation time (fill time). By controlling the fill time of ions into the trap, where the flux of ions into the trap is known or can be approximated, the total number of ions accumulated in the ion trap can be controlled. As such, in some embodiments, the isolated ionised oligonucleotides may be accumulated within the secondary ion trap using automatic gain control (AGC) techniques. In some preferred embodiments, an accumulation time of the isolated ionised oligonucleotides may be based on an in silico determination of the ion current.

Once accumulated in the ion trap, ions within the trap can be ejected into the mass analyser. The ions may be ejected from the ion trap in a direction orthogonal to the axis of the trap (orthogonal ejection), for example by applying one or more suitable DC voltages to the ion trap. The ions may be injected into the mass analyser via one or more lenses and a deflector electrode.

The method further comprises a fourth step (iv) of obtaining a mass spectrum by detecting the isolated ionised oligonucleotides of each m/z window using a high-resolution mass spectrometer (HRMS) having a resolving power of greater than 30,000. That is, after isolation of ionised oligonucleotides of a specific m/z range, the ionised oligonucleotides are passed to a mass analyser of a high-resolution mass spectrometer. Preferably, the high-resolution mass spectrometer has a resolving power of greater than 60,000, preferably greater than 100,000 (generally specified as FWHM at m/z 200).

A high-resolution mass spectrometer with high resolving capability is preferred to measure the signal intensity resulting from each unique mass of oligonucleotide within each “bin” of a given oligonucleotide length. This is achieved by the resolving power of the mass analyser which allows for a precise measurement of the ion mass. However, isobaric oligonucleotides are structural isomers and therefore have the same exact mass and are generally not differentiable by such direct mass spectrometry but will contribute to the signal intensity of the mass peak (mass envelope) of a given mass.

Any high-resolution mass spectrometer may be used for the method of the present invention.

Preferably the high-resolution mass spectrometer comprises a time-of-flight (ToF) mass analyser, or an ion trap mass analyser, for detection of the ionised oligonucleotides in step (iv), preferably a multi-reflecting time-of-flight mass analyser or an orbital trapping mass analyser. Suitable examples which are commercially available include those which comprise an Orbitrap™ or Astral™ mass analyser, as available from Thermo Fisher Scientific.

As described in, for example, GB2618673A, an ion trap mass analyser can be any suitable type of ion trap having one or more detection electrodes configured to detect image current signals from ions accumulated within the ion trap. Examples of suitable such ion traps include, for example, multi-reflection and multi-deflection electrostatic traps and time-of-flight analysers, orbital traps (including of Cassini type) with one or multiple inner electrodes, linear and 3D traps with RF trapping, and so on. The ion trap may comprise detection circuitry configured to provide an output signal based on the detected image current signals. An image current may be detected using a differential amplifier connected to a first outer detection electrode and a second outer detection electrode of the trap.

The ion trap mass analyser may have a trapping volume therein in which ions may be trapped. The ion trap mass analyser may be an electrostatic trap, such as an electrostatic orbital trap. The orbital ion trap mass analyser may have an inner electrode arranged along an axis and two outer detection electrodes spaced apart along the axis and surrounding the inner electrode. When the ion trap mass analyser is operated in a mass analysis mode of operation, ions trapped within the ion trap may oscillate with a frequency which may depend on their mass-to-charge ratio and which can be detected using image current detection in which the pair of outer electrodes function as detection electrodes to detect an image current induced by the oscillation of the ions in the trapping volume and thereby provide a detected signal. The detected signal can be processed using Fourier transformation to obtain a mass spectrum of ions within the trap. The ions may perform substantially harmonic oscillations along the axis in an electrostatic field whilst orbiting around the inner electrode. As described above, the orbital ion trap may be an Orbitrap™ mass analyser, further details of which may be found in, for example, in U.S. Pat. No. 5,886,346A.

As will be appreciated, a mass spectrometer can include any number of one or more additional components such as ion optical devices. For example, the instrument may include one or more ion transfer stage(s) arranged between any of the components, e.g. including an atmospheric pressure interface and/or one or more ion guides, lenses and/or other ion optical devices configured such that some or all of the ions can be transmitted appropriately through the instrument. The ion transfer stage(s) may include any suitable number and configuration of ion optical devices, for example optionally including one or more ion guides, lenses and/or other ion optical devices.

For example, in embodiments the mass spectrometer may optionally include a collision or reaction cell downstream of the secondary ion trap. Where the collision or reaction cell is present, ions collected in the secondary ion trap can either be ejected orthogonally to the primary ion trap mass analyser without entering the collision or reaction cell, or the ions can be transmitted axially to the collision or reaction cell for processing before returning the processed ions to the ion trap for subsequent orthogonal ejection to the mass analyser ion trap. The processing may comprise, for example, fragmenting the ions by collisions with a collision gas and/or a reagent in the collision cell, or further cooling the ions by collisions with a gas at a lower energy that does not fragment the ions. That is, in some embodiments, between the isolation and detection steps (iii) and (iv), the method further comprises fragmentation of the isolated ionised oligonucleotides. Such a method may be referred to as tandem mass spectrometry (or MS/MS). It is however advantageous that the present method does not require such additional steps in the analysis providing a simple, fast and accurate confirmation.

The mass spectrometer is generally under the control of a control unit, such as an appropriately programmed computer, which controls the operation of various components of the spectrometer and, for example, sets the voltages to be applied to the various components of the spectrometer. The control unit may also receive and process data from various components including the detector(s), e.g. perform Fourier transformation on detected signals. The control unit may be configured, amongst other things, to determine the settings (e.g. secondary ion trap fill time, etc.) for the injection of ions into the mass analyser for analytical scans.

The mass spectrometer may be operated such that successive batches of ions from the ion source are each accumulated in and analysed by the ion trap mass analyser. Each batch of ions is firstly accumulated in the secondary ion trap, and then the accumulated ions are injected into the mass analyser. It is desirable that each batch of ions analysed by the mass analyser includes as many ions as possible, so as to improve the statistics of the mass spectrum. However, undesirable space charge effects can occur at relatively high ion concentrations and can limit mass resolution and mass accuracy. Therefore, the total number of ions accumulated in the ion trap is controlled to optimise the number of ions injected into the mass analyser to be below, but as close as possible to, a limit for the mass analyser, such as the space-charge limit for the mass analyser. The total number of ions accumulated in the ion trap may also or instead be controlled to be below a limit for the ion trap such as the space-charge limit for the ion trap. Typically, between 1×10⁴ and 1×10⁶ elementary charges should be stored, such as between 1×10⁵ and 5×10⁵.

Therefore, as described above, embodiments can use so-called automatic gain control (AGC) techniques to precisely control the total number of ions accumulated in the ion trap. These techniques typically rely on an accurate and reliable real-time estimation of the present ion current J or ion flux being received by the ion trap. Then, by controlling the filling time Tf of the ion trap, the total number of ions or the total amount of charge accumulated in the trap (and injected into the mass analyser) can be suitably controlled.

Thus, a batch of ions that is to be injected into the mass analyser is first stored in the secondary ion trap, where its total charge Q=J*Tf can be determined by the secondary ion trap filling time Tf during which the secondary ion trap receives an ion current J from the ion source. The accumulation time (e.g. fill time) of ions in the trap is adjusted based on an estimation of the present ion current J or ion flux to control the total number of ions accumulated in the ion trap, or advantageously may be based on an in silico determination of the ion current given the expected/calculated number of ions within a given m/z window. In this way, the total number of ions N injected into the mass analyser per scan can then be controlled in order to achieve an appropriate signal-to-noise ratio while avoiding adverse space-charge effects caused by the analyser overfilling.

Preferably, the multi-reflecting time-of-flight mass analyser is configured in a multi-pass mode of operation. GB2619766A, for example, relates to methods of analysing labelled analyte molecules and/or ions using time-of-flight (ToF) mass analysers and describes the use of multi-reflecting time-of-flight mass analysers suitable for the present method.

The multi-reflection ToF mass analyser may be of the tilted-mirror type described in US2015/0028197A1, though other variations could be used. In such an embodiment, the ToF mass analyser includes an extraction trap, whereby ions may be accumulated and cooled in the extraction trap. The extraction trap may incorporate two trapping regions, one at a relatively higher pressure for rapid ion cooling, and a second low pressure region for ion extraction. Ions are cooled in the high-pressure region and then transferred to the low-pressure region, where they are pulse ejected into the ToF analyser via a pair of deflectors. As will be appreciated, the same considerations about the space charge limit described above also apply to this extraction trap. Ions oscillate between a pair of mirrors, which are tilted relative to one another so that the ion path is slowly deflected and redirected back to a detector. Correcting stripe electrodes counter the loss of ion focus otherwise induced by the non-parallelism of the mirrors.

The multi-reflection time-of-flight analyser includes a pair of ion mirrors that are spaced apart and face each other in a first direction X. The ion mirrors are elongated along an orthogonal drift direction Y between a first end and a second end. The extraction trap is arranged at one end of the analyser. Ions are accumulated in the extraction trap, before being injected into the space between the ion mirrors. Ions may be injected from the extraction trap with a relatively small injection angle or drift direction velocity, creating a zigzag ion trajectory, whereby different oscillations between the mirrors are separate in space.

One or more lenses and/or deflectors may be arranged along the ion path, between the ion source and the ion mirror first encountered by the ions. For example, an injection deflector may be arranged along the ion path, between the ion source and the ion mirror first encountered by the ions. Other arrangements would be possible. In general, the one or more lenses and/or deflectors may be configured to suitably condition, focus and/or deflect the ion beam, i.e. such that it is caused to adopt the desired trajectory through the analyser. The analyser may also include another deflector, which is arranged along the ion path, between the ion mirrors. The deflector may be arranged approximately equidistant between the ion mirrors, along the ion path after its first ion mirror reflection, and before its second ion mirror reflection.

The analyser also includes a detector. The detector may be any suitable ion detector configured to detect ions and record an intensity and time of arrival associated with the arrival of ion(s) at the detector. Suitable detectors include, for example, one or more conversion dynodes, optionally followed by one or more electron multipliers, and the like.

In its “normal” mode of operation, ions are injected from the extraction trap into the space between the ion mirrors, in such a way that the ions adopt a zigzag ion path having plural reflections between the ion mirrors in the X direction, whilst: (a) drifting along the drift direction Y from the deflector towards the opposite (second) end of the ion mirrors, (b) reversing drift direction velocity in proximity with the second end of the ion mirrors, and then (c) drifting back along the drift direction Y to the deflector. The ions can then be caused to travel from the deflector to the detector for detection.

The ions mirrors may both be tilted with respect to the X and/or drift Y direction. It would instead be possible for only one of the ion mirrors to be tilted, and, for example, for the other one of the ion mirrors to be arranged parallel to the drift Y direction. In general, the ion mirrors are a non-constant distance from each other in the X direction along most or all of their lengths in the drift direction Y. The drift direction velocity of ions towards the second end of the ion mirrors is opposed by an electric field resulting from the non-constant distance of the two mirrors from each other, and this electric field causes the ions to reverse their drift direction velocity in proximity with the second end of the ion mirrors and drift back along the drift direction towards the deflector.

In embodiments, the analyser further comprises a pair of correcting stripe electrodes. Ions travelling down the drift length are slightly deflected with each pass through the mirrors and the additional stripe electrodes are used to correct for the time-of-flight error created by the varying distance between the mirrors. For example, the stripe electrodes may be electrically biased such that the period of ion oscillation between the mirrors is substantially constant along the whole of the drift length (despite the non-constant distance between the two mirrors from). The ions eventually find themselves reflected back down the drift space and focused at the detector.

Alternatively, the ion mirrors may be parallel to each other. In these embodiments, in order to cause the ions to reverse their drift direction velocity in proximity with the second end of the ion mirrors and drift back along the drift direction towards the deflector, the analyser may include another deflector at the second end of the ion mirrors.

In these embodiments, a lens can be included in the injection deflector and/or in the deflector. Thus, the ion beam is allowed to expand a short way into the analyser before meeting a long-focus lens, which has the effect of focussing the ion beam along its length. The lens may be an elliptical drift focusing (converging) lens mounted within the deflector. The second deflector, which may also include a lens, is used to reverse the beam direction whilst maintaining control of focal properties. Further detail of the single-lens type multireflection time-of-flight mass analyser is given in GB2580089A.

In such analysers, the ion beam is allowed to spread out relatively broadly (in the drift direction Y) for most of its flight path. This is in contrast, for example, with multi-reflecting time-of-flight (MR-ToF) mass analysers which use a set of periodic lenses to focus the ion beam along its entire flight path. A significant advantage of allowing the ion beam to spread out broadly for most of its flight path is that space charge effects are reduced, which can be a significant problem for time-of-flight analysers.

In embodiments, the MR-ToF mass analyser is operable in a multi-pass “zoom” mode of operation. In this mode of operation, ions are made to make multiple cycles within the analyser in the drift direction Y. Increasing the number of cycles N increases the length of the ion path that ions take within the analyser (between the injector and the detector), thereby increasing the resolution of the analyser.

The deflector at the front of the analyser, which is normally used to reduce the injection angle and/or optimise the number of oscillations within a single drift pass, may (also) be used to reverse the drift direction velocity of the ions such that the ions are caused to complete a further cycle through the analyser. Thus, in a multi-pass “zoom” mode of operation, ions are caused to complete plural (N) cycles within the analyser, where in each cycle the ions drift in the drift direction Y from the deflector (or entrance lens) towards the opposite (second) end of the ion mirrors, and then back to the deflector (or entrance lens). In each cycle, the ions also complete plural reflections between the ion mirrors in the X direction. Thus, in each cycle, the ions adopt a zigzag ion path through the space between the ion mirrors.

An initial cycle may be initiated by injecting the ions from the extraction trap into the space between the ion mirrors. The ions may be reflected in one of the ion mirrors and may then travel to the deflector. An appropriate (e.g. relatively small) voltage may be applied to the deflector such that the ions are caused to exit the deflector in a direction towards the second end of the ion mirrors. Upon existing the deflector, the ions adopt a zigzag ion path having plural reflections between the ion mirrors in the direction X whilst: (a) drifting along the drift direction Y from the deflector towards the second end of the ion mirrors, (b) reversing drift direction velocity in proximity with the second end of the ion mirrors, and (c) drifting back along the drift direction Y to the deflector.

After the ions have completed this initial cycle, each further cycle is initiated by using the deflector to reverse the drift direction velocity of the ions (in proximity with the first end of the ion mirrors). To do this, an appropriate voltage may be applied to the deflector that causes ions to leave the deflector with a drift direction velocity opposite to the drift direction velocity with which the ions originally entered the deflector. This voltage may be applied during a time period in which it is expected that the ions will arrive back at the deflector. Suitable deflection voltages to reverse the drift direction of the ions are of the order of hundreds of volts.

The deflector may be used to reverse the drift direction velocity of the ions one or more times. Thus, the method may comprise causing the ions to complete plural (N) cycles within the analyser, where the first cycle is initiated by injecting the ions into the space between the ion mirrors, and after the ions have completed the first cycle, each further cycle may be initiated by using the deflector to reverse the drift direction velocity of the ions.

After the ions have completed the desired (plural) number (N) of cycles within the analyser, the ions are allowed to travel from the deflector to the detector for detection. To do this, an appropriate voltage may be applied to the deflector such that the ions are caused to exit the deflector in a direction towards the detector. The ions may be reflected in (the other) one of the ion mirrors before travelling to (and being detected by) the detector.

In this zoom mode, the analyser in effect “zooms in” on a narrower m/z region of the m/z spectrum, i.e. the zoom mode provides very high resolution but only for a narrow region of m/z (because it is necessary to avoid faster ions “lapping” slower ions), and so it is particularly suitable for analysing each m/z window of interest with high resolution.

The isolated ionised oligonucleotides of each m/z window may be detected in step (iv) individually during a scan. That is, in some embodiments, each target window is analysed in its own separate mass analysis “scan”. Each scan typically takes around 500 milliseconds (where each scan may therefore be of a window encompassing one oligonucleotide length). Thus 10 scans, for example, would require 5 seconds, and 20 scans would require 10 seconds.

Alternatively, the isolated ionised oligonucleotides of two or more m/z windows may be detected in step (iv) simultaneously during a scan. That is, it would be possible to use “multiplexing”, where the mass filter described herein is used to select two or more oligonucleotides of different length to target during a single scan (i.e. the same single fill of the ion trap, ejection into the mass analyser, and subsequent mass analysis). This can be done by moving the mass filter between the different target windows during the same single fill time, or operating the mass filter with a “comb”-like transmission profile.

The sample consumption rate during the ionisation can be tailored and therefore minimised to the type of the applied mass analyser. MALDI is a discontinuous desorption type of ionisation technique, or may be considered quasi-continuous at higher repetition rates, such as above 3 kHz, though laser pulses can still be interrupted on demand. Such discontinuously running laser ionisation can be tailored in a way that the laser desorption events are synchronised with the ion filling process of the secondary ion trap.

If higher resolving power or MS/MS fragmentation is further implemented confirming of the identity of the analysed sample, the time needed for a longer transient acquisition (and therefore analysis) at higher resolving power, and the additional time required for running the MS/MS fragmentation event, can be extended while the event of desorption and ionisation is interrupted. In addition, the direct sample introduction without tedious chromatographic separation offers the advantage that the sample analysis can be repeated several times in sequence with minimal additional time effort.

The method further comprises a fifth step (v) of confirming the sequence of the nucleic acid by comparison of the mass spectrum obtained in step (iv) against a mass spectrum determined in silico following the same hydrolytic cleavage of the nucleic acid as in step (i), wherein the confirmation comprises comparison of the signal intensities of one or more mass envelopes resulting from the ionised oligonucleotides. The mass envelopes being well separated in the mass space by the use of a high resolution mass spectrometer allows for the accurate determination of signal intensity of individual oligonucleotides which is essential for an accurate comparison. The separation which may be achieved by the present method has limitations with previous methods, such as those based on axial ToF.

For the expert in the field of mass spectrometric analysis it is well known that ToF analysis operating in axial mode offers a best achievable mass accuracy of between 5 and 50 ppm for smaller molecules (where the analyte is anticipated to be less fragile during the ionisation). If vacuum MALDI desorption and ionisation is applied for the ToF analysis of oligonucleotides at lower pressure, the mass accuracy of such larger biomolecules can be worse and within a range of 100 ppm. In general, ToF running in axial mode is known to be not as accurate as a multi-reflecting time-of-flight mass analyser or an orbital trapping mass analyser, each of which are known to provide mass accuracy at ppm mass accuracy level (e.g. from 1 to 5 ppm).

Another disadvantage of the prior art methods of vacuum MALDI ToF analysis is the expected moderate resolution particularly in the axial mode of operation ranging between 500 and 2,000. Resolution in mass spectrometry is a measure of the instrument's ability to distinguish between two adjacent mass peaks. It is typically defined as the ratio of the mass of an ion to the difference in mass between two ions that can be distinguished from each other. The most common way to express this is as the full width at half maximum (FWHM) resolution. The resolution is defined by the determined mass-to-charge divided by the full width at half maximum.

Higher resolution means the instrument can distinguish between ions with very small differences in mass, which is crucial for accurate mass determination and for resolving complex mixtures. The impact of higher resolution becomes more important if the analysed mixture is more complex. The complexity increases for a higher number of atoms contributing to the chemical structure of the analysed sample if the atoms fall into the category of multiple isotopes such as carbon, nitrogen, oxygen and hydrogen.

Oligonucleotides, fall into the category of complex chemical structures, because oligonucleotides, which are short sequences of nucleotides, exhibit highly complex isotopic patterns, especially as the length of the nucleotide sequence exceeds three. This complexity arises due to the presence of multiple isotopes of the constituent elements (such as carbon, nitrogen, oxygen, and hydrogen) in the nucleotides, which combine in various ways to form numerous isotopic variants.

The final isotope distribution of a molecule, such as an oligonucleotide, is often referred to as the “mass envelope”. The mass envelope represents the range of masses and their relative abundances resulting from the different isotopic compositions of the molecule. This term is frequently used in mass spectrometry to describe the pattern of peaks corresponding to the isotopic variants of a molecule.

A positive confirmation may therefore be achieved by matching the signal intensities of each mass envelope within a given set of windows, ideally all windows. A match may be considered where the signal intensity is within ±10% of the expected value, preferably within ±5%.

As will be appreciated, each ion will comprise a natural abundance of isotopes giving rise to a “mass envelope” consisting of individual “mass peaks” arising from specific isotopes of the ionised oligonucleotide (for example, those generally resulting from ¹²C and ¹³C, and ¹⁴N and ¹⁵N isotopes). The signal intensity is based on the total area of the mass envelope, and it is generally sufficient for the integration of the areas associated with the most abundant mass peaks. This may generally be the most intense 3 to 6 mass peaks of a mass envelope. In an essentially equivalent alternative, this may be based on a signal-to-noise (S/N) threshold whereby mass peaks with an S/N of greater than 3:1, or even greater than 2:1, are included. In embodiments, the confirmation described herein in step (v) may further comprise comparison of the isotopic pattern of the one or more, or all/each of the mass envelopes. Such an analysis constitutes a subsidiary confirmation step to provide greater confidence to the confirmation achieved by the signal intensity comparison thereby aiding in the reduction of false positives.

Each nucleotide consists of three main components: (i) the nitrogenous base, (ii) the pentose sugar and (iii) the phosphate diester. The pentose sugar and the phosphate diester form the backbone of a single-stranded DNA or RNA, while the nitrogenous base, attached to the pentose sugar, functions as the coding region of the biomolecule. To confirm the identity of DNA or RNA, the mass envelope of an oligonucleotide must take all three main components into account.

The chemical formula and, therefore, the nominal molecular mass of the nitrogenous base of natural DNA and RNA are set out in table 1 below:

TABLE 1

| | | Nominal |
|---|---|---|
| | Molecular | molecular mass |
| Nitrogenous base | formula | (Da/amu) |
| Guanine | C₅H₅N₅O | 151 |
| Adenine | C₅H₅N₅ | 135 |
| Thymine (including 5-methyluracil and | C₅H₆N₂O₂ | 126 |
| N1-methylpseudouracil) | | |
| Uracil | C₄H₄N₂O₂ | 112 |
| Cytosine | C₄H₅N₃O | 111 |

The chemical composition of oligonucleotides having identical length (in terms of the number of nucleotides) generally differ as a result of the different masses of the bases within the sequence. Because the deoxyribose (for DNA) or ribose for (RNA) and the phosphate diester are a connecting link with constant molecular mass, the oligonucleotides with different numbers of nucleotides are separated from each other in the mass domain by (i) the mass of the nitrogenous base; (ii) the mass of the five-carbon sugar: deoxyribose for DNA, or ribose for RNA; and (iii) the mass of the phosphate diester.

The nominal molecular mass of ribose (C₅H₁₀O₅) is 150 Da (or amu), and the nominal molecular mass of deoxyribose is 134 Da. That of phosphoric acid (H₃PO₄) is 98 Da. The nominal molecular mass of the combination of sugar and phosphate (i.e. (ii) and (iii)) may be calculated as follows (taking into account the loss of water (H₂O) which has a nominal molecular mass of 18 Da):
RNA: 150+98−18=230 Da
DNA: 134+98−18=214 Da

As such, the nominal molecular mass of the DNA nucleotides may be calculated as follows (again taking into account the loss of water):
Deoxyguanosine monophosphate (C₁₀H₁₄N₅O₇P): 214+151−18=347 Da
Deoxyadenosine monophosphate (C₁₀H₁₄N₅O₆P): 214+135−18=331 Da
Deoxythymidine monophosphate (C₁₀H₁₅N₂O₈P): 214+126−18=322 Da
Deoxycytidine monophosphate (C₉H₁₄N₃O₇P): 214+111−18=307 Da

If MALDI ionisation is applied for the analysis of hydrolytic cleavage products of DNA or RNA, predominantly singly charged ions are produced. Under such a condition, the oligonucleotides with different nucleotide lengths are well separated in mass, because the molecular mass of the sugar and phosphate (which can be seen as mass spacer between the nitrogenous bases) is much greater than the mass differences that exist between the different nitrogenous bases.

If a base-specific hydrolytic cleavage is applied with a G-specific DNAse or RNAse or other type of G-specific hydrolytic cleavage reaction, then the abundance of guanine nitrogenous base within the cleavage products is limited to a low number or ideally one. For such G-specific hydrolytic cleavage products, the maximum mass difference between two oligonucleotides having the same length is defined by the difference in mass of one sequence in which the bases consist of adenine and another sequence in which the bases consist of cytosine (and assuming that the same number, i.e. only one, guanine is present in each sequence). This is due to adenine and cytosine having the highest and lowest molecular masses, respectively, with those of thymine and uracil falling in between these extremes.

Such maximum mass difference in between two cleavage products can be expected to be rare and therefore can be seen as an unlikely assumption because such poly-A or poly-C regions can be expected to be a non-coding regime.

The nominal mass difference between A and C is 135−111=24 Da, equivalent to the mass represented by the sum of one additional carbon atom and two additional nitrogen atoms, minus a single oxygen atom.

Up to the oligonucleotide sequence length of 13 nucleotides, the mass of the phosphodiester and the (deoxy-)ribose is expected to be higher than the calculated maximum mass difference of the nitrogenous bases if all 13 bases are either A or C. In other words, a modern type of mass selecting filter can easily separate the individual lengths of any mixture of oligonucleotide sequences below the sequence length of 13 in the assumption most of the ionised oligonucleotides are singly charged.

In some preferred embodiments, the confirmation is achieved in step (v) by an exact match of the quantised signal intensities of each mass envelope resulting from ionised oligonucleotides having from 8 to 12 nucleotides. That is, the confirmation (i.e. positive or negative) comprises comparison of the quantised signal intensities of each mass envelope resulting from ionised oligonucleotides having from 8 to 12 nucleotides for an exact match. Therefore, such a match provides a positive confirmation, and any differences between the spectra provides a negative confirmation.

The confirmation may comprise comparison of the mass envelopes resulting from oligonucleotides having more than 12 nucleotides, though above about 16, there are generally very few which may then lead to error. Similarly, one particular benefit of the invention is a comparison of quantised signal intensities such that a lower limit of 5 or 6 oligonucleotides is generally preferred since the natural abundances of individual species are unlikely to be too great allowing for quantised comparisons. Whilst it is generally preferred to include a greater number of m/z windows of oligonucleotides to provide greater statistical significance to the confirmation, such limits mean that an exact match within the 6 to 14 nucleotide windows may be preferred.

In other words, the range of m/z windows of oligonucleotides that are to be included in the comparison are preferably those in which, once normalised to mass envelopes of unit signal intensity (i.e. 1), all of the other mass envelopes within a given isolation window have a signal intensity than is ten times greater or less (i.e. 10 or less). Each of the mass envelopes in a given m/z window will then be quantised in the sense of having a signal intensity that is assigned the closest integer of from 1 to 10, and this process advantageously allows for a fast and accurate comparison with in silico data and an exact match of the integer values.

For the longer oligonucleotides, it is naturally the case that statistically, these are generally all unique fragments with repeated fragments less likely. The presence of repeated fragments are still acceptable since there are unlikely to be more than 2 (and even less likely more than 3), such that the signal intensity can still be quantised (normalised) to allow for a simple comparison, particularly within the range of from 1 to 10 as described above. The mass spectrum displays a unique fingerprint in this region, primarily as a result of the existence of isobaric oligonucleotides. These are structural isomers which have the same exact mass and thus contribute to the signal of the same mass envelope. By way of example only, two isobaric 6mer oligonucleotides include CCC CAG and CCA CCG.

Below lengths of 5 or 6 oligonucleotides (i.e. lengths of 2 to 4 or 5), there are likely to be a significant number of repeated oligonucleotides (i.e. non-unique fragments) which contribute to the signal intensity of a mass envelope at specific masses. As such, the signal intensity is typically much larger and tends to become more continuous with differences arising from, for example, the presence of isobaric masses, more challenging to detect. In any event, these may also be compared and matched for additional certainty, and a match for such mass envelopes may be considered where the signal intensity is within ±10% of the expected value, preferably within ±5%.

It is also a particular advantage of the method described herein that is does not require the use of liquid chromatography (LC) or gas chromatography (GC). Thus, the method provides a fast and accurate confirmation of a nucleic acid sequence. The method can permit a high throughput comparative sequence conformation of nucleic acids, such as RNA vaccines, within acquisition durations of less than 60 seconds per sample which avoids the multiple hours required for chromatographic separations.

In embodiments, the confirmation in step (v) may also further comprise comparison of the collision cross-sections (CCS) as determined from time-domain transient decay of a detection signal from each isobaric ionised oligonucleotide. Such values can be used to help identify and differentiate between isobaric oligonucleotides. US2023/0136166A1 discloses suitable methods for determining a measure of a rate of decay of an ion sample undergoing mass analysis in a mass analyser with image current detection, which may be used in the present method. Embodiments use a measure of the signal intensity, such as S/N ratio (SNR), for intensity dependent (e.g. SNR and/or m/z dependent) corrections of decay times, which correspond to the CCS. CCS can be measured by the mobility of ions, such as may be determined in ion mobility drift cells. If a compound shares the same chemical formula or composition of nucleotides, the 3-dimensional structure may differ, and thus their drift. Ultimately, a sufficiently high resolution of applied ion mobility device instrument is required if an isobaric molecule with two or more different structures is to be separated in the mobility domain.

In some embodiments, the confirmation in step (v) further comprises confirmation of a length of a poly-A tail of the nucleic acid, which may be present as an in-tact oligonucleotide where a suitable digesting enzyme is selected which avoids cleavage of phosphate bonds between adenosine bases (e.g. the G-specific RNase T1).

The features described herein in respect of the first aspect apply equally to the features of this further aspect and vice versa. Differences in the two experimentally determined mass spectra can be used to identify structural differences in the nucleic acid with respect to the reference (whose sequence is known or predetermined), and/or changes in the m/z ratio of the mass envelope may be used to identify those sequences which have been modified with respect to the reference. As such, the method still allows for confirmation of the nucleic acid sequence with reference to the parent nucleic acid. Equally, the reference nucleic acid may be analysed in silico in accordance with the first aspect.

In particular, the determination of the sequence may preferably be achieved in step (v) by comparison of the quantised signal intensities of each mass envelope resulting from isobaric ionised oligonucleotides having from 8 to 12 nucleotides, preferably 6 to 14 nucleotides. As will be appreciated, an exact match may be achieved for those parts of the nucleic acid which remain unchanged, and the differences may be used (for example by cross-referencing masses who signal intensities have grown or shrunk) to determine the nature of the structural changes.

The present method for confirming the sequence identity is intended to apply the method for the accurate mass determination to in depth screen and assign an exchange of a single or several nucleotides within the analysed oligonucleotide after hydrolytic cleavage.

For the expert in the field, it is well known that a G-specific hydrolytic cleavage is suitable for identifying the nucleotide exchange from A, C, T(or U) to G, such as is already described in the aforementioned Nucleic Acids Research 2003, 31(9), e47. This is because the additional G nucleotide is adding another cleavage position resulting in two shorter oligonucleotides.

Table 2 below shows the calculated accurate mass differences of the three different possibilities if a single nucleotide is replaced in the oligonucleotide without considering the exchange of the nucleotide G. For example, the exchange from T to A results in an increased exact mass of 9.01157 Da. Such increased exact mass results from the addition of three nitrogen atoms (3×14.00307) and the loss of two oxygen atoms (2×15.99492) as well as one hydrogen atom (1×1.007825).

TABLE 2

| | Carbon | Hydrogen | Nitrogen | Oxygen | Delta mass |
|---|---|---|---|---|---|
| Exchange | 12.0000 | 1.007825 | 14.00307 | 15.99492 | (Da) |
| T → A | 0 | −1 | 3 | −2 | 9.011567 |
| C → T | 1 | 1 | −1 | 1 | 14.999666 |
| C → A | 1 | 0 | 2 | −1 | 24.011233 |

As will be appreciated, a reverse exchange, such as from A to T, results in a negative mass difference (delta mass). The presented method of analysing the mass differences, if polymorphism is investigated, should not be limited to the nucleotides A, T, and C, but may also be used for the analysis of RNA nucleotides A, C, and U. For recent mRNA vaccine synthesis, the N-methylated pseudouridine (m1ψ) plays an important role. The chemical formula of the associated methylated uracil base is identical to the chemical formula of thymidine. Thus the same calculations as those presented above for A, C and T, apply equally for A, C and m1ψ. In other words, the method for confirming the sequence may be one for a nucleic acid comprised of G, A, C, and U nucleotides; G, A, C, and T nucleotides; or G, A, C, and m1ψ nucleotides.

The advantage of the present method for applying the accurate mass in comparison to previous methods where the nominal mass is considered, can be demonstrated by consideration of exchange of one, two, three, four, fix or six nucleotides within an oligonucleotide,

If the less accurate nominal mass differences of 9 Da (T→A), 15 Da (C→T) or 24 Da (C→A) are applied, then several combinations of nucleotide exchanges result in the same nominal molecular mass difference although the exchange of the nucleotide themselves within two sequences are different.

For example, if within an oligonucleotide sequence with a composition of (a) six nucleotides 3×C and 3×T (such as CCC TTT) is replaced by (b) another oligonucleotide sequence having 6×A (AAA AAA), the nominal mass difference is 99 Da. The same nominal mass difference of 99 Da will occur if 6×C (CCC CCC) is replaced by 5×T and 1×A (TTT TTA). The present method allows for the differentiation between two different narrow mass differences which are given for the two exchanges, for example (a) a nucleotide exchange of CCC TTT→AAA AAA which is calculated to have an accurate mass difference of 99.0684 Da or (b) a nucleotide exchange of CCC CCC→TTT TTA which is calculated to have an accurate mass difference 99.009563 Da.

In total, 127 possible exchanges are possible without considering repetitions. For 76 of the possible exchanges, the nominal mass difference is not unique (that is, there are 38 pairs of exchanges with the same nominal mass difference). As such, for about 60% of all cases, the less accurate nominal mass determination cannot differentiate in between two different possibilities of nucleotide exchange. This is ultimately a hindrance for the subsequent analysis and structural confirmation.

Consequently, a mass difference of a multiple of (i.e. a linear combination of) 9.01157 Da, 14.99967 Da and/or 24.01123 Da between a mass signal in the analysed sample when compared to the reference (e.g. an in silico calculated mass spectrum of hydrolytic cleavage products or an experimental spectrum) may be used to identify at least one nucleotide polymorphism in a cleavage product of the analysed sample, and preferably up to six nucleotide polymorphisms (though there is no specific upper limit). That is, such mass difference(s) between identified mass signals (e.g. as a result of a change in signal intensity) may be used to identify the replacement of one or more of A↔T, C↔T and/or C↔A in the process of confirming the sequence of a nucleic acid.

As will be appreciated, the above discussed accurate mass differences in between the three different nucleotides A, C and T are identical if mRNA is analysed where the all Us are replaced by N-methylated pseudouridine (m1ψ).

Table 3 below identifies all of the possible mass differences wherein up to six nucleotides are exchanged, together with the associated nominal and accurate mass differences.

TABLE 3

| | | | mass | mass | unique | delta | delta |
|---|---|---|---|---|---|---|---|
| Index | before | after | (before) | (after) | (nominal) | nominal | accurate |
| 1 | CCCCCC | AAAAAA | 1830.2477 | 1974.3151 | yes | −144 | −144.0674 |
| 2 | CCCCCC | TAAAAA | 1830.2477 | 1965.3036 | yes | −135 | −135.0558 |
| 3 | CCCCCT | AAAAAA | 1845.2474 | 1974.3151 | yes | −129 | −129.0677 |
| 4 | CCCCCC | TTAAAA | 1830.2477 | 1956.292 | yes | −126 | −126.0443 |
| 5 | CCCCC | AAAAA | 1525.2064 | 1645.2626 | yes | −120 | −120.0562 |
| 6 | CCCCCC | TTTAAA | 1830.2477 | 1947.2804 | yes | −117 | −117.0327 |
| 7 | CCCCTT | AAAAAA | 1860.2471 | 1974.3151 | yes | −114 | −114.0681 |
| 8 | CCCCC | TAAAA | 1525.2064 | 1636.251 | yes | −111 | −111.0446 |
| 9 | CCCCCC | TTTTAA | 1830.2477 | 1938.2689 | yes | −108 | −108.0211 |
| 10 | CCCCT | AAAAA | 1540.2061 | 1645.2626 | yes | −105 | −105.0565 |
| 11 | CCCCC | TTAAA | 1525.2064 | 1627.2395 | yes | −102 | −102.033 |
| 12 | CCCTTT | AAAAAA | 1875.2467 | 1974.3151 | no | −99 | −99.0684 |
| 13 | CCCCCC | TTTTTA | 1830.2477 | 1929.2573 | no | −99 | −99.00956 |
| 14 | CCCC | AAAA | 1220.1651 | 1316.2101 | yes | −96 | −96.04493 |
| 15 | ccccc | TTTAA | 1525.2064 | 1618.2279 | yes | −93 | −93.02146 |
| 16 | CCCTT | AAAAA | 1555.2058 | 1645.2626 | no | −90 | −90.05683 |
| 17 | CCCCCC | TTTTTT | 1830.2477 | 1920.2457 | no | −90 | −89.998 |
| 18 | CCCC | TAAA | 1220.1651 | 1307.1985 | yes | −87 | −87.03336 |
| 19 | CCTTTT | AAAAAA | 1890.2464 | 1974.3151 | no | −84 | −84.06873 |
| 20 | CCCCC | TTTTA | 1525.2064 | 1609.2163 | no | −84 | −84.0099 |
| 21 | CCCT | AAAA | 1235.1648 | 1316.2101 | yes | −81 | −81.04527 |
| 22 | CCCC | TTAA | 1220.1651 | 1298.1869 | yes | −78 | −78.0218 |
| 23 | CCTTT | AAAAA | 1570.2054 | 1645.2626 | no | −75 | −75.05717 |
| 24 | CCCCC | TTTTT | 1525.2064 | 1600.2048 | no | −75 | −74.99833 |
| 25 | CCC | AAA | 915.12386 | 987.15756 | yes | −72 | −72.0337 |
| 26 | CTTTTT | AAAAAA | 1905.2461 | 1974.3151 | no | −69 | −69.06907 |
| 27 | CCCC | TTTA | 1220.1651 | 1289.1754 | no | −69 | −69.01023 |
| 28 | CCTT | AAAA | 1250.1645 | 1316.2101 | no | −66 | −66.0456 |
| 29 | ACCCCC | TTTTTT | 1854.259 | 1920.2457 | no | −66 | −65.98676 |
| 30 | CCC | TAA | 915.12386 | 978.14599 | yes | −63 | −63.02213 |
| 31 | CTTTT | AAAAA | 1585.2051 | 1645.2626 | no | −60 | −60.0575 |
| 32 | CCCC | TTTT | 1220.1651 | 1280.1638 | no | −60 | −59.99866 |
| 33 | CCT | AAA | 930.12353 | 987.15756 | yes | −57 | −57.03403 |
| 34 | TTTTTT | AAAAAA | 1920.2457 | 1974.3151 | no | −54 | −54.0694 |
| 35 | CCC | TTA | 915.12386 | 969.13443 | no | −54 | −54.01056 |
| 36 | CTTT | AAAA | 1265.1641 | 1316.2101 | no | −51 | −51.04593 |
| 37 | ACCCC | TTTTT | 1549.2177 | 1600.2048 | no | −51 | −50.9871 |
| 38 | CC | AA | 610.08257 | 658.10504 | yes | −48 | −48.02247 |
| 39 | TTTTT | AAAAA | 1600.2048 | 1645.2626 | no | −45 | −45.05784 |
| 40 | CCC | TTT | 915.12386 | 960.12286 | no | −45 | −44.999 |
| 41 | CTT | AAA | 945.12319 | 987.15756 | no | −42 | −42.03437 |
| 42 | AACCCC | TTTTTT | 1878.2702 | 1920.2457 | no | −42 | −41.97553 |
| 43 | CC | TA | 610.08257 | 649.09347 | yes | −39 | −39.0109 |
| 44 | TTTT | AAAA | 1280.1638 | 1316.2101 | no | −36 | −36.04627 |
| 45 | ACCC | TTTT | 1244.1764 | 1280.1638 | no | −36 | −35.98743 |
| 46 | CT | AA | 625.08224 | 658.10504 | yes | −33 | −33.0228 |
| 47 | TTTTTT | AAAAAC | 1920.2457 | 1950.3039 | no | −30 | −30.05817 |
| 48 | CC | TT | 610.08257 | 640.08191 | no | −30 | −29.99933 |
| 49 | TTT | AAA | 960.12286 | 987.15756 | no | −27 | −27.0347 |
| 50 | AACCC | TTTTT | 1573.2289 | 1600.2048 | no | −27 | −26.97586 |
| 51 | C | A | 305.04129 | 329.05252 | yes | −24 | −24.01123 |
| 52 | TTTTT | AAAAC | 1600.2048 | 1621.2514 | no | −21 | −21.0466 |
| 53 | ACC | TTT | 939.13509 | 960.12286 | no | −21 | −20.98776 |
| 54 | TT | AA | 640.08191 | 658.10504 | no | −18 | −18.02313 |
| 55 | AAACCC | TTTTTT | 1902.2814 | 1920.2457 | no | −18 | −17.9643 |
| 56 | C | T | 305.04129 | 320.04095 | yes | −15 | −14.99967 |
| 57 | TTTT | AAAC | 1280.1638 | 1292.1988 | no | −12 | −12.03504 |
| 58 | AACC | TTTT | 1268.1876 | 1280.1638 | no | −12 | −11.9762 |
| 59 | T | A | 320.04095 | 329.05252 | yes | −9 | −9.011567 |
| 60 | TTTTTT | AAAACC | 1920.2457 | 1926.2927 | no | −6 | −6.046936 |
| 61 | AC | TT | 634.09381 | 640.08191 | no | −6 | −5.988099 |
| 62 | TTT | AAC | 960.12286 | 963.14633 | no | −3 | −3.023468 |
| 63 | AAACC | TTTTT | 1597.2401 | 1600.2048 | no | −3 | −2.964631 |
| 64 | TTTTT | AAACC | 1600.2048 | 1597.2401 | no | 3 | 2.964631 |
| 65 | AAC | TTT | 963.14633 | 960.12286 | no | 3 | 3.023468 |
| 66 | TT | AC | 640.08191 | 634.09381 | no | 6 | 5.988099 |
| 67 | AAAACC | TTTTTT | 1926.2927 | 1920.2457 | no | 6 | 6.046936 |
| 68 | A | T | 329.05252 | 320.04095 | yes | 9 | 9.011567 |
| 69 | TTTT | AACC | 1280.1638 | 1268.1876 | no | 12 | 11.976198 |
| 70 | AAAC | TTTT | 1292.1988 | 1280.1638 | no | 12 | 12.035035 |
| 71 | T | C | 320.04095 | 305.04129 | yes | 15 | 14.999666 |
| 72 | TTTTTT | AAACCC | 1920.2457 | 1902.2814 | no | 18 | 17.964297 |
| 73 | AA | TT | 658.10504 | 640.08191 | no | 18 | 18.023134 |
| 74 | TTT | ACC | 960.12286 | 939.13509 | no | 21 | 20.987765 |
| 75 | AAAAC | TTTTT | 1621.2514 | 1600.2048 | no | 21 | 21.046602 |
| 76 | A | C | 329.05252 | 305.04129 | yes | 24 | 24.011233 |
| 77 | TTTTT | AACCC | 1600.2048 | 1573.2289 | no | 27 | 26.975864 |
| 78 | AAA | TTT | 987.15756 | 960.12286 | no | 27 | 27.034701 |
| 79 | TT | CC | 640.08191 | 610.08257 | no | 30 | 29.999332 |
| 80 | AAAAAC | TTTTTT | 1950.3039 | 1920.2457 | no | 30 | 30.058169 |
| 81 | AA | CT | 658.10504 | 625.08224 | yes | 33 | 33.0228 |
| 82 | TTTT | ACCC | 1280.1638 | 1244.1764 | no | 36 | 35.987431 |
| 83 | AAAA | TTTT | 1316.2101 | 1280.1638 | no | 36 | 36.046268 |
| 84 | TA | CC | 649.09347 | 610.08257 | yes | 39 | 39.010899 |
| 85 | TTTTTT | AACCCC | 1920.2457 | 1878.2702 | no | 42 | 41.97553 |
| 86 | AAA | CTT | 987.15756 | 945.12319 | no | 42 | 42.034367 |
| 87 | TTT | CCC | 960.12286 | 915.12386 | no | 45 | 44.998998 |
| 88 | AAAAA | TTTTT | 1645.2626 | 1600.2048 | no | 45 | 45.057835 |
| 89 | AA | CC | 658.10504 | 610.08257 | yes | 48 | 48.022466 |
| 90 | TTTTT | ACCCC | 1600.2048 | 1549.2177 | no | 51 | 50.987097 |
| 91 | AAAA | CTTT | 1316.2101 | 1265.1641 | no | 51 | 51.045934 |
| 92 | TTA | CCC | 969.13443 | 915.12386 | no | 54 | 54.010565 |
| 93 | AAAAAA | TTTTTT | 1974.3151 | 1920.2457 | no | 54 | 54.069402 |
| 94 | AAA | CCT | 987.15756 | 930.12353 | yes | 57 | 57.034033 |
| 95 | TTTT | CCCC | 1280.1638 | 1220.1651 | no | 60 | 59.998664 |
| 96 | AAAAA | CTTTT | 1645.2626 | 1585.2051 | no | 60 | 60.057501 |
| 97 | TAA | CCC | 978.14599 | 915.12386 | yes | 63 | 63.022132 |
| 98 | TTTTTT | ACCCCC | 1920.2457 | 1854.259 | no | 66 | 65.986763 |
| 99 | AAAA | CCTT | 1316.2101 | 1250.1645 | no | 66 | 66.0456 |
| 100 | TTTA | CCCC | 1289.1754 | 1220.1651 | no | 69 | 69.010231 |
| 101 | AAAAAA | CTTTTT | 1974.3151 | 1905.2461 | no | 69 | 69.069068 |
| 102 | AAA | CCC | 987.15756 | 915.12386 | yes | 72 | 72.033699 |
| 103 | TTTTT | CCCCC | 1600.2048 | 1525.2064 | no | 75 | 74.99833 |
| 104 | AAAAA | CCTTT | 1645.2626 | 1570.2054 | no | 75 | 75.057167 |
| 105 | TTAA | CCCC | 1298.1869 | 1220.1651 | yes | 78 | 78.021798 |
| 106 | AAAA | CCCT | 1316.2101 | 1235.1648 | yes | 81 | 81.045266 |
| 107 | TTTTA | CCCCC | 1609.2163 | 1525.2064 | no | 84 | 84.009897 |
| 108 | AAAAAA | CCTTTT | 1974.3151 | 1890.2464 | no | 84 | 84.068734 |
| 109 | TAAA | CCCC | 1307.1985 | 1220.1651 | yes | 87 | 87.033365 |
| 110 | TTTTTT | CCCCCC | 1920.2457 | 1830.2477 | no | 90 | 89.997996 |
| 111 | AAAAA | CCCTT | 1645.2626 | 1555.2058 | no | 90 | 90.056833 |
| 112 | TTTAA | CCCCC | 1618.2279 | 1525.2064 | yes | 93 | 93.021464 |
| 113 | AAAA | CCCC | 1316.2101 | 1220.1651 | yes | 96 | 96.044932 |
| 114 | TTTTTA | CCCCCC | 1929.2573 | 1830.2477 | no | 99 | 99.009563 |
| 116 | AAAAAA | CCCTTT | 1974.3151 | 1875.2467 | no | 99 | 99.0684 |
| 117 | TTAAA | CCCCC | 1627.2395 | 1525.2064 | yes | 102 | 102.03303 |
| 118 | AAAAA | CCCCT | 1645.2626 | 1540.2061 | yes | 105 | 105.0565 |
| 119 | TTTTAA | CCCCCC | 1938.2689 | 1830.2477 | yes | 108 | 108.02113 |
| 120 | TAAAA | CCCCC | 1636.251 | 1525.2064 | yes | 111 | 111.0446 |
| 121 | AAAAAA | CCCCTT | 1974.3151 | 1860.2471 | yes | 114 | 114.06807 |
| 122 | TTTAAA | CCCCCC | 1947.2804 | 1830.2477 | yes | 117 | 117.0327 |
| 123 | AAAAA | CCCCC | 1645.2626 | 1525.2064 | yes | 120 | 120.05617 |
| 124 | TTAAAA | CCCCCC | 1956.292 | 1830.2477 | yes | 126 | 126.04426 |
| 125 | AAAAAA | CCCCCT | 1974.3151 | 1845.2474 | yes | 129 | 129.06773 |
| 126 | TAAAAA | CCCCCC | 1965.3036 | 1830.2477 | yes | 135 | 135.05583 |
| 127 | AAAAAA | CCCCCC | 1974.3151 | 1830.2477 | yes | 144 | 144.0674 |

Three exemplary nucleic acids were analysed in silico and the mass spectra determined by calculation of the expected signal intensities. Specifically, the three nucleic acids analysed were the Comirnaty BNT162b2 “Original” DS mRNA vaccine against SARS-CoV-2, as well as the subsequent BNT162b2s04 “Delta” and BNT162b2s05 “Omicron” variants, and via an in silico digest with the G-specific RNase T1, this produced 1,062 cleavage products for the Original and Omicron, and 1,065 cleavage products for Delta.

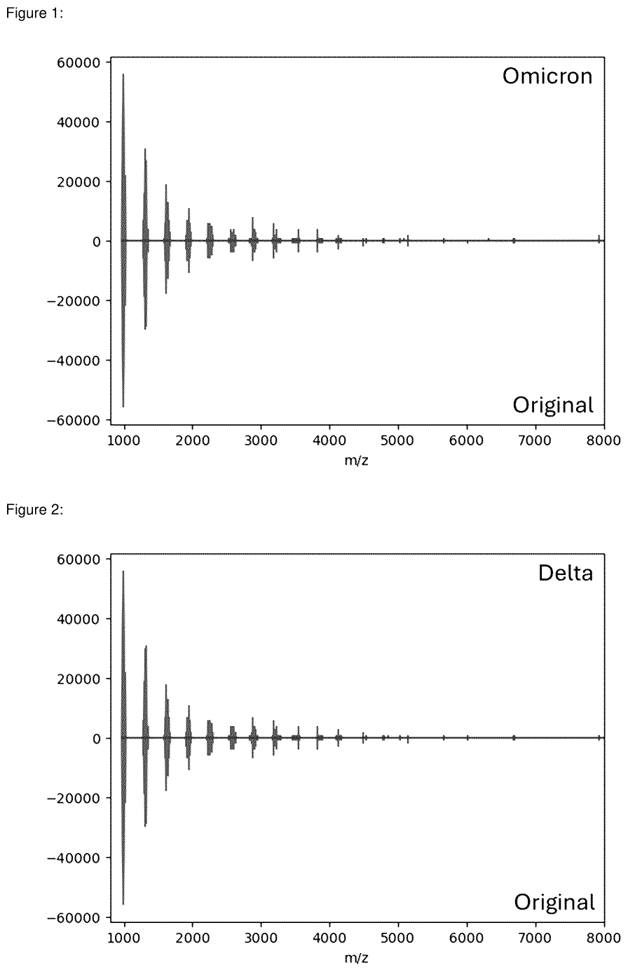

FIGS. 1-7 provides various plots of the calculated data, normalised signal intensity on the y-axis (with mass envelopes normalised to an intensity of 1,000 for a one unit peak), at different ranges of m/z ratio on the x-axis. In all Figures, the data for the Original “parent” nucleic acid is plotted as negative intensity values to provide a direct illustration of the difference between the Omicron (FIGS. 1 and 3-7) and Delta (FIGS. 2 and 8) variants, whose data is provided as positive intensity values.

FIGS. 1 and 2 illustrate the in silico calculation of the signal intensities of the plurality of oligonucleotides obtained from digestion of both the Comirnaty BNT162b2 Original DS vaccine and the BNT162b2s05 Omicron variant, or BNT162b2s04 Delta variant, respectively. The calculation spans the m/z range of 800 to 8,000, and demonstrates the binning that is observed of oligonucleotides of different lengths into mass separated windows. Specifically, the range spans 3mers at about m/z 1,000 up to 25mers at about m/z 8,000. Above m/z 8,000 (not shown) are the poly-A tail cleavage products of lengths 32 and 73 for each mRNA.

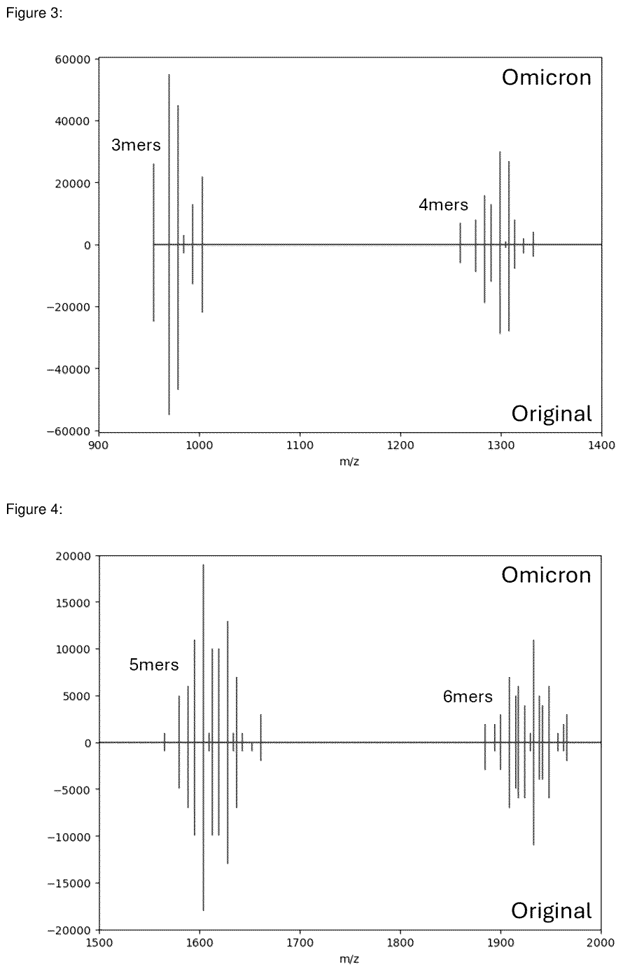

FIG. 3 provides a close-up view of the data of FIG. 1 within the m/z range of 900 to 1,400, and shows the signal intensities calculated for the 3mers and 4mers of the Original and Omicron variant. As can be seen, the intensity of many individual mass envelopes is in excess of 20,000. FIG. 4 provides a close-up view of the data of FIG. 1 within the m/z range of 1,500 to 2,000, and shows the signal intensities calculated for the 5mers and 6mers of the Original and Omicron variant.

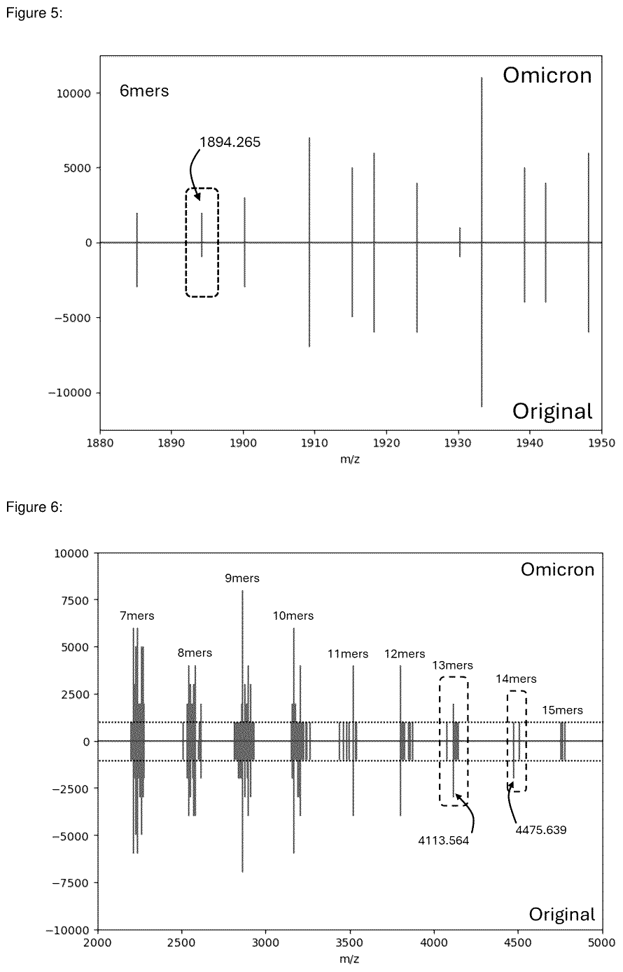

FIG. 5 provides a close-up view of the data of FIG. 1 (and FIG. 4) within the m/z range of 1,880 to 1,950, i.e. that specifically of a portion of the 6mers. The signal at m/z 1,894.265 is highlighted due to the difference in intensity calculated for the two mRNAs. For the Original, there is only one unique cleavage product, specifically CCC CAG (@3387) providing a mass envelope of unit signal intensity. The signal for the Omicron variant is twice as intense due to the contribution of two cleavage products of isobaric mass, specifically CCA CCG (@2084) and CCC CAG (@3387). All of the other signals have quantised signal intensities ranging from 1 to 11, with further differences identifiable between the signals at 1,885.254, 1,924.264, and 1,939.264.

FIG. 6 provides a close-up view of the data of FIG. 1 within the m/z range of 2,000 to 5,000, and shows the signal intensities calculated for the 7mers to 15mers of the Original and Omicron variant. The horizontal dotted lines provide an indication of the unit signal intensities of ±1,000. All of the calculated intensities for the mass envelopes associated with the 7mers to 15mers are below 10,000, and are thus quantised within the range of from 1 to 10.

The signal at m/z 4,113.564 is highlighted due to the difference in intensity calculated for the two expected cleavage products of the two mRNAs. For the Original, there three unique cleavage products, specifically C TAC TTC CCA CTG (@1517), T CCT CCA ACT TCG (@2952), and C CCA CTT TCC TAG (@3313) providing a mass envelope having a quantised signal intensity of 3. The signal for the Omicron variant is two-thirds as intense due to the contribution from only two cleavage products of isobaric mass, specifically C TAC TTC CCA CTG (@1508), and C CCA CTT TCC TAG (@3304) providing a mass envelope having a quantised signal intensity of 2. Similarly, the signal at m/z 4,475.639 has a quantised signal intensity of 2 for the Original due to two unique fragments, specifically TC CAA CAT CAT CAG (@345), and TC TAC TAC CAC AAG (@481), whereas the signal for the Omicron variant has a quantified signal intensity of 1 due to the one unique fragment of TC CAA CAT CAT CAG (@339) for this mass.

These differences may be used to identify differences between variants of nucleic acids to confirm the sequence of a nucleic acid, or serve to demonstrate the comparison which may be made between an experimentally determined mass spectrum and that calculated in silico.

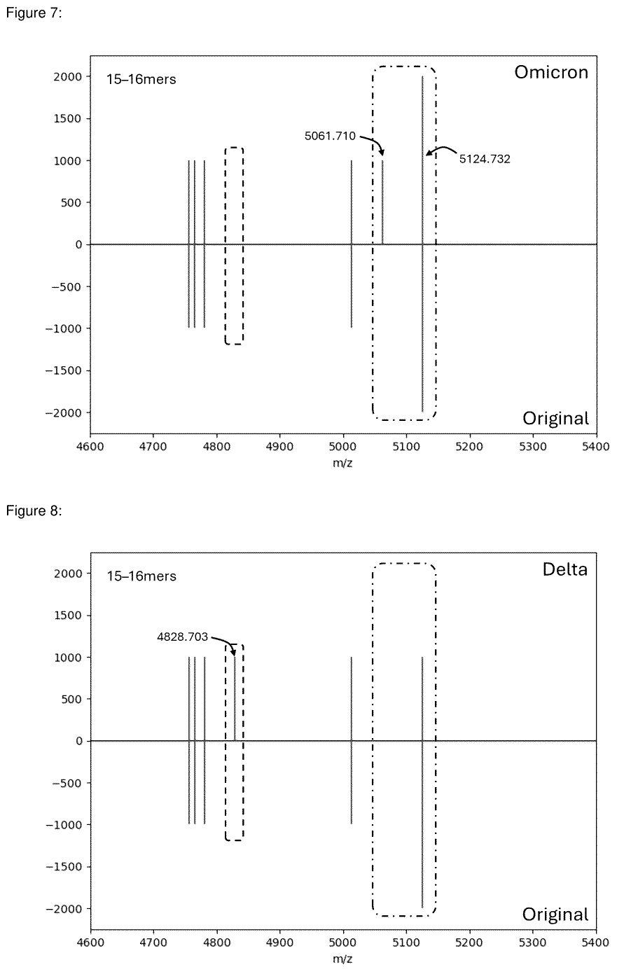

FIGS. 7 and 8 provide a close-up views within the m/z range of 4,600 to 5,400, of the data of FIGS. 1 and 2, respectively. These show the signal intensities calculated for the 15mers to 16mers of the Original and Omicron variant (FIG. 7) and the Original and Delta variant (FIG. 8). It can be seen that the signal at m/z 4,828.703 is absent in both the Original and the Omicron variant, but is present in the Delta variant due to the fragment CAA CTA CAA TTA CAG (@1388). Similarly, it can be seen that the signal at m/z 5,061.710 is absent in both the Original and the Delta variant, but is present in the Omicron variant due to the fragment C ACA CCC CTA TCA TCG (@657). Finally, whilst there is no difference between the signal at m/z 5,124.732 in the Original and the Omicron variant, both having a quantised signal intensity of 2 arising from the unique isobaric fragments C AAC TAC AAT TAC CTG (Original @1394; Omicron @1385), and A ACT TCA ACT TCA ACG (Original @1671; Omicron @1662), only the fragment A ACT TCA ACT TCA ACG (@1665) is present in the Delta variant.

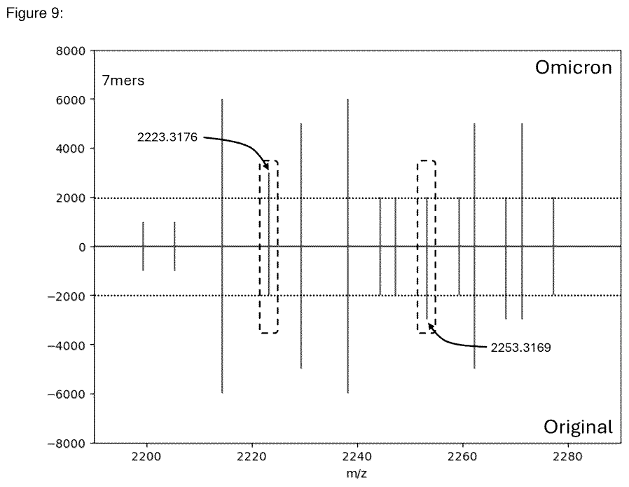

The benefit of applying accurate mass determination is demonstrated by the close-up view in FIG. 9 showing the expected signal intensities of the 7mers cleavage products. The accurate mass difference of the present method is confirming the replacement within two cleavage products that two T-nucleotides are exchanged by two C-nucleotides. The intensity for the signal at m/z 2253.3169 is 50% greater for the Original mRNA when compared to the Omicron variant (i.e. a quantised signal intensity ratio of 3:2). Additionally, the intensity for the signal at m/z 2223.3176 is 50% greater for the Omicron variant when compared to the Original. The resulting accurate mass difference between both signals of 29.9993 Da thus provides independent verification that such mass difference can only be the exchange of two Ts by two As. This Example therefore demonstrates the identification of a variation (or what may be described a mutation) in the mRNA sequence of the cleavage product C TAT CAG (@1565) in the Original to C CAC CAG (@1554) in the Omicron variant by identification of a mass difference that is a multiple of 14.99967 Da (specifically two times T→A).

These Figures demonstrate the particular benefit that may be achieved in the simplicity and accuracy of sequence confirmation in an exact match of the quantised signal intensities of each mass envelope resulting from ionised oligonucleotides having from 8 to 12 nucleotides, more preferably the larger range of from 6 to 14 nucleotides for greater certainty.

As used herein, the singular form of “a”, “an” and “the” include plural references unless the context clearly dictates otherwise. The use of the term “comprising” is intended to be interpreted as including such features but not excluding other features and is also intended to include the option of the features necessarily being limited to those described. In other words, the term also includes the limitations of “consisting essentially of” (intended to mean that specific further components can be present provided they do not materially affect the essential characteristic of the described feature) and “consisting of” (intended to mean that no other feature may be included such that if the components were expressed as percentages by their proportions, these would add up to 100%, whilst accounting for any unavoidable impurities), unless the context clearly dictates otherwise.

Numerical lower and upper limits of features described herein may preferably be combined to provide a closed range.

The foregoing detailed description has been provided by way of explanation and illustration, and is not intended to limit the scope of the appended claims. Many variations of the presently preferred embodiments illustrated herein will be apparent to one of ordinary skill in the art, and remain within the scope of the appended claims and their equivalents.

For the avoidance of doubt, the entire contents of all documents acknowledged herein are incorporated herein by reference.

1. A method for confirming the sequence of a nucleic acid, the method comprising:
(i) hydrolytic cleavage of predetermined phosphodiester bonds in the nucleic acid to produce a plurality of oligonucleotides;
(ii) ionising the plurality of oligonucleotides to provide a gaseous phase of ionised oligonucleotides, wherein a majority of the ionised oligonucleotides are singly charged;
(iii) isolating the ionised oligonucleotides using a plurality of mass to charge ratio (m/z) windows;
(iv) detecting the isolated ionised oligonucleotides of each m/z window using a high-resolution mass spectrometer (HRMS) having a resolving power of greater than 30,000 at m/z 200, thereby obtaining a mass spectrum;
(v) confirming the sequence of the nucleic acid by comparison of the mass spectrum obtained in step (iv) against a mass spectrum determined in silico following the same hydrolytic cleavage of the nucleic acid as in step (i), wherein the confirmation comprises comparison of the signal intensities of one or more mass envelopes resulting from the ionised oligonucleotides.

2. The method according to claim 1, wherein the confirmation is achieved in step (v) by an exact match of the quantised signal intensities of each mass envelope resulting from ionised oligonucleotides having from 8 to 12 nucleotides, preferably 6 to 14 nucleotides.

3. The method according to claim 1, wherein the nucleic acid is DNA or RNA, preferably mRNA.

4. The method according to claim 1, wherein the hydrolytic cleavage of phosphodiester bonds in step (i) is performed by digestion of the nucleic acid with an endonuclease.

5. The method according to claim 4, wherein the endonuclease is RNase T1.

6. The method according to claim 1, wherein at least 90%, preferably substantially all, of the ionised oligonucleotides are singly charged.

7. The method according to claim 1, wherein the plurality of oligonucleotides is ionised in step (ii) by MALDI, photoionisation or DART.

8. The method according to claim 7, wherein the plurality of oligonucleotides is ionised in step (ii) by atmospheric pressure MALDI.

9. The method according to claim 8, wherein the plurality of oligonucleotides is ionised in step (ii) by liquid atmospheric pressure MALDI.

10. The method according to claim 1, wherein the nucleic acid comprises at least 1,000 nucleotides, preferably from 1,000 to 4,000 nucleotides.

11. The method according to claim 1, wherein the high-resolution mass spectrometer has a resolving power of greater than 60,000, preferably greater than 100,000.

12. The method according to claim 1, wherein the isolation in step (iii) is performed using a mass filter, preferably a quadrupole mass filter.

13. The method according to claim 1, wherein each of the plurality of m/z windows corresponds to a number of nucleotides present in each oligonucleotide, preferably wherein each window does not overlap.

14. The method according to claim 1, wherein the isolated ionised oligonucleotides of each m/z window are detected in step (iv) individually during a scan.

15. The method according to claim 1, wherein the isolated ionised oligonucleotides of two or more m/z windows are detected in step (iv) simultaneously during a scan.

16. The method according to claim 1, wherein the high-resolution mass spectrometer comprises a time-of-flight (ToF) mass analyser, or an ion trap mass analyser, for detection of the ionised oligonucleotides in step (iv), preferably a multi-reflecting time-of-flight mass analyser or an orbital trapping mass analyser.

17. The method according to claim 16, wherein the high-resolution mass spectrometer comprises a multi-reflecting time-of-flight mass analyser configured in a multi-pass mode of operation.

18. The method according to claim 1, wherein between steps (iii) and (iv), the method comprises accumulating the isolated ionised oligonucleotides within an ion trap arranged upstream of the mass analyser and periodically passing the accumulated ionised oligonucleotides from the ion trap to the mass analyser for detection of the ionised oligonucleotides.

19. The method according to claim 18, wherein the isolated ionised oligonucleotides are accumulated within the secondary ion trap using automatic gain control (AGC) techniques.

20. The method according to claim 19, wherein an accumulation time of the isolated ionised oligonucleotides is based on an in silico determination of the ion current.

21. The method according to claim 1, wherein between steps (iii) and (iv), the method further comprises fragmentation of the isolated ionised oligonucleotides.

22. The method according to claim 1, wherein the method does not comprise liquid chromatography (LC) or gas chromatography (GC).

23. The method according to claim 1, wherein the confirmation in step (v) further comprises comparison of the collision cross-sections (CCS) as determined from time-domain transient decay of a detection signal from each isobaric ionised oligonucleotide.

24. The method according to claim 1, wherein the confirmation in step (v) further comprises comparison of the isotopic pattern of the one or more mass envelopes.

25. The method according to claim 1, wherein the confirmation in step (v) further comprises identification of at least one nucleotide polymorphism by a mass difference of a multiple of 9.01157 Da, 14.99967 Da and/or 24.01123 Da between a mass signal in the analysed sample when compared to the reference.

26. The method according to claim 1, wherein the confirmation in step (v) comprises comparison of the mass spectrum obtained in step (iv) against a database of mass spectra, each determined in silico following the same predetermined hydrolytic cleavage in step (i) of a further nucleic acid.

27. The method according to claim 1, wherein the confirmation in step (v) further comprises confirmation of a length of a poly-A tail of the nucleic acid.

28. A method for confirming the sequence of a nucleic acid, the method comprising:
(i) hydrolytic cleavage of predetermined phosphodiester bonds in the nucleic acid to produce a plurality of oligonucleotides;
(ii) ionising the plurality of oligonucleotides to provide a gaseous phase of ionised oligonucleotides, wherein a majority of the ionised oligonucleotides are singly charged;
(iii) isolating the ionised oligonucleotides using a plurality of mass to charge ratio (m/z) windows;
(iv) detecting the isolated ionised oligonucleotides of each m/z window using a high-resolution mass spectrometer (HRMS) having a resolving power of greater than 30,000 at m/z 200, thereby obtaining a mass spectrum;
(v) confirming the sequence of the nucleic acid by comparison of the mass spectrum obtained in step (iv) against a mass spectrum obtained by duplicating steps (i) to (iv) with a reference nucleic acid, wherein the confirmation comprises comparison of the signal intensities of one or more mass envelopes resulting from the ionised oligonucleotides.
